(19) United States
(12) Patent Application Publication
(10) Pub. No.: US 2018/0021374 A1
(43) Pub. Date: Jan. 25, 2018

(54) Antibacterial Clay Compositions for Use as a Topical Ointment

(71) Applicant: Bishopsgate Exploration Ltd., Winnipeg (CA)

(72) Inventor: Tim Tuba, Winnipeg (CA)

(21) Appl. No.: 15/216,940

(22) Filed: Jul. 22, 2016

(51) Int. Cl. A61K 33/26; A61K 9/00; A61K 9/06; A61K 9/14; A61K 33/06

(52) U.S. Cl. CPC A61K 33/26; A61K 9/14; A61K 33/06; A61K 9/06; A61K 9/0014

(57) ABSTRACT

The present invention is directed to synthetic bactericidal compositions having clay like properties and a method of using these compositions to topically treat bacterially-caused skin infections and skin diseases. The compositions within the scope of the invention comprise a bactericidal effective amount of a reducing agent, such as pyrite, marcasite, pyrrhotite, FeS2, FeS, FeSO4—or other reducing agents having like properties, and a natural clay or clay mineral and/or synthetic clay or clay mineral, or other suitable materials having clay-like properties with a high (>10) or low (<5) pH. The synthetic bactericidal compositions are synthesized by adding the reducing agent to the either high (>10) or low (<5) pH clay or clay mineral. It is the presence of both the reducing agent and the high (>10) or low (<5) pH clay of said compositions which renders them bactericidal.

### FIELD OF INVENTION

The present invention relates to synthetic antibacterial compositions having clay-like properties and a method of using these compositions to topically treat bacterially-caused skin infections and skin diseases.

This invention is an improvement of the compositions and method shown in US Published application 2013/0004544 (Metge et al) published Jan. 3 2013 which is now abandoned. The full disclosure of this published application is incorporated herein by reference.

### BACKGROUND OF THE INVENTION

The present invention relates to synthetic antibacterial compositions having clay-like properties and a method of using these compositions to topically treat bacterially-caused skin infections and skin diseases. The compositions within the scope of the invention comprise a bactericidal effective amount of a reducing agent, such as pyrite, marcasite, pyrrhotite, FeS2, FeS, FeSO4, or other reducing agents having like properties, and a natural clay or clay mineral and/or a synthetic clay or clay mineral, or other suitable materials having clay-like properties. Divalent iron within the structure of a clay mineral itself may also serve as a reducing agent. It is the presence of the reducing agent in said compositions that renders them antibacterial. The clays or clay minerals serve as a vehicle within which the reducing agent is dispersed, as a dilutent to the reducing agent, and also as an adsorbent and low permeability barrier in use of the composition. These compositions are hydrated so as to form a paste, which is then to be applied to the affected area for treatment. Compositions within the scope of the present invention are suitable to topically treat infections and skin diseases caused by one or more types of bacteria, including antibiotic-resistant bacteria.

Antibiotics are available to treat various types of skin infections. Their route of administration for such treatments can vary. Some are prescribed to be taken orally, some to be applied topically, while others to be administered intravenously. The present invention relates to an antibacterial composition that is to be applied topically to an affected area. In particular, the present invention relates to synthetic anti-bacterial clay (ABC) compositions.

Studies have been conducted exploring the potential use of natural clays in the topical treatment of bacterial infections. For example, certain natural clays have been used in the Ivory Coast to treat Buruli ulcer: see http:/fifthkingdom.net/BuruliBusters/default.htm. Although researchers have identified a few natural clays as having bactericidal activity, these studies have not been conclusive as to how or why these clays are effective. Additionally, these studies did not provide definitive guidance as to how one having ordinary skill in the art would conclusively be able to select or identify suitable antibacterial natural clays, or, for that matter, synthesize suitable antibacterial clay-like or clay containing compositions for topical treatment of bacterial infections. Applicants are not aware of any specific guidance present in the art that might direct one skilled in the art to do so. Mechanistically, it has been unclear how these clays produce bactericidal activity.

According to Williams et al., [Williams et al., “Chemical and Mineralogical Characteristics of French Green Clays Used for Healing,” Clays and Clay Minerals, Vol. 56, No. 4, 437-452 (2008)], the current accepted treatment of M. ulcerans ulcers larger than 5 cm is surgical excision, limb amputation, and/or subsequent skin grafting.

Certain natural clays have been used in the Ivory Coast to treat Buruli ulcer, a flesh-eating disease caused by Mycobacterium ulcerans. This has been documented in O'Hanlon, “Medicinal Clays May Heal Ulcers,” News in Science, 26 Oct. 2007. This article reports a French clay identified as Agricur* as effective against this flesh-eating disease in Africa's Ivory Coast, and that an interdisciplinary team of microbiologists and mineralogists was attempting to discover how the clay cures. (*Per applicants, although the article references this clay as “Agricur,” this clay is correctly referred to as “Argicur,” carrying the company/supplier name, Argicur Inc. Le Buisson de Cadouin, France.) The article further indicates that two mineralogically-similar clays had different antibacterial activity. Therefore, based on this article, it appears uncertain which specific clay compositions would be effective for the treatment of bacterial infections. Research was initiated into determining what makes one clay toxic to bacteria and another harmless. The article further indicates that several well-known, pathogenic bacteria Salmonella typhimurium, Streptococcus sp., Escherichia coli and Pseudomonas sp. were exposed to the clays; bacterial cultures lost 90-99% (1-2 log unit loss) viability within 24 hours of exposure to French Argicur clays. This was in contrast to only 10-40% reduced viability (0.2-1 log unit loss) caused by other clays or sterile sand. Based on the article, the mechanism as to how the clay works is uncertain. It is also uncertain, based on this article, as to what would make a specific clay more suitable for such treatment, and what would motivate one having ordinary skill in the art to select one clay over another for such treatment.

Consistent with this article are the results and conclusions set forth in Williams et al., “Kiler Clays! Natural antibacterial clay minerals,” Mineralogical Society Bulletin, 139: 3-8 (2004). The article references and explores the effect on bacteria of two French clays from two different suppliers. The authors reference therein that “[i]t was immediately apparent that one of the clay samples was not effective in killing Mycobacterium, but was more suited to promotion of skin granulation after the Mycobacterium were killed. These observations remain unexplained.” Further, based on the teachings therein, it appeared that one of the clays (Argiletz) apparently kills Mycobacterium ulcerans (in human trials); but, in vitro, this same clay enhanced E. coli, whereas the Argicur clay killed E. coli. Based on the article, much needs to be explored to determine what properties of clay, if any, may render it antibacterial. The authors of the article indicate that they are exploring numerous variables, such as, trace element exchange, surface free energy potential, pH, oxidation state, and how these vary with mineral morphology. No specific information is identified as to what properties and/or composition a clay must have in order to exhibit antibacterial (bactericidal) properties.

Haydel et al., “Broad-spectrum in vitro antibacterial activities of clay minerals against antibiotic-susceptible and antibiotic-resistant bacterial pathogens,” J. of Antimicrobial Chemotherapy (2008) 61, 353-361, reported that two iron-rich clay minerals, which are similar in major phases and bulk chemistry, have striking and opposite effects on bacterial populations, ranging from enhanced microbial growth to complete growth inhibition. The article states that “of the six independent clay samples collected from the French suppler and tested against various bacteria . . . only CsAg02 displayed antibacterial effects . . . there is not a single component of CsAg02 clay (e.g. transition metals) that stands out as an obvious antibacterial agent, so it may be a fortuitous combination of factors (multiple components) responsible for the Inhibitory property.” (at p. 359)

Through their extensive research, applicants did, however, identify a property which renders the natural anti-bacterial clays tested (i.e., the Argicur used in the Ivory Coast and Pyroclay) antibacterial. In particular, applicants identified that the presence of particular reducing agents (pyrite in these), in particular amounts, and in fine particle form, is what renders these natural clays antibacterial. These reducing agents were found to be absent in clays found to not have bactericidal properties. It is the identification of these reducing agents that lead to the present invention. Applicants are now able to artificially produce an antibacterial (bactericidal) composition having clay-like properties referred to herein as synthetic antibacterial composition or synthetic bactericidal composition. These compositions may have some advantages over simply using a natural antibacterial clay in that they can be customized to illicit desired properties i.e., purity level. In particular, the present invention relates to synthesized antibacterial compositions, containing a clay or clay mineral and a bactericidal effective amount of a reducing agent, that Applicants believe may be used to topically treat most, if not all, bacterial skin infections, including those caused by antibiotic resistant bacteria. In use, the composition within the scope of the invention is hydrated to form a paste, which is applied to the affected area. Although Brunet de Courssou reported that treatment of Buruli ulcer with clay was found to be painful, it is applicants' belief that some patients may find the treatment reasonably painless; some may even find it soothing.

The term “antibacterial” is used in the title herein, because an antibacterial clay or clay mineral may either kill bacteria, and therefore be “bactericidal,” or render them bacteriostatic, which means that the bacteria cannot grow or reproduce. The terms antibacterial and bactericidal are used interchangeably herein. Many agents that are bactericidal are also antimicrobial, which means that the antimicrobial agents may attack bacteria, viruses, protozoa, fungus, etc.

### SUMMARY OF THE INVENTION

The invention relates to synthetic antibacterial compositions to topically treat bacterially-caused skin infections and skin diseases. These synthetic compositions have the properties of clay (clay-like properties), and contain therein an antibacterial effective amount of a reducing agent in a clay. These compositions may be prepared by adding an antibacterial effective amount of a reducing agent (for example, pyrite) to a clay or clay mineral with an either high (>10) or low (<5) pH, wherein the synergistic effect of both reducing agent and clay renders the composition antibacterial.

The compositions comprise a bactericidal effective amount of a reducing agent, such as pyrite, marcasite, pyrrhotite, FeS2, FeS, FeS04 or other reducing agents having like properties, and a natural clay or clay mineral and/or a synthetic clay or clay mineral or other suitable materials having clay-like properties and a high (>10) or low (<5) pH. The clays, by themselves, can serve as a moderate bactericidal agent due to the low or high pH. However, it is the presence of the reducing agent in combination with the high (>10) or low (<5) pH clay in the compositions that renders them antibacterial. The compositions herein are suitable to reduce or eliminate bacterial growth caused by one or more types of bacteria.

The presence of particular reducing agents (pyrite for example), in particular amounts, and in fine particle form, together with high (>10) or low (<5) pH clays or clay minerals can render the clays antibacterial. It is the identification of both these reducing agents and high (>10) or low (<5) pH clays that lead to the present invention.

The arrangement herein provides an antibacterial (bactericidal) composition having clay-like properties referred to herein as synthetic antibacterial composition or synthetic bactericidal composition. These compositions may have some advantages over simply using a natural antibacterial clay in that they can be customized to provide desired properties i.e., purity level. In particular, the present invention relates to synthesized antibacterial compositions, containing a clay or clay mineral and an effective amount of a reducing agent that may be used as a topical ointment.

Preferably the present method of producing the various compositions involves quarrying the clay, air drying the clay until it has a moisture content of 20% to 25% by weight. The clay is subsequently crushed and Introduced to a rotary dryer and blended with the remaining ingredients of the composition. The composition remains in the rotary dryer until it has a moisture content between 10% and 15% by weight. Water, or other suitable pharmaceutically acceptable aqueous liquids (e.g., Inert solution), are added to a composition in sufficient amounts to create a paste. The hydrated composition is then applied topically to the site of the bacterial skin infection.

The Invention further relates to synthetic antibacterial clays or clay minerals, which may be prepared by synthesizing high (>10) or low (<5) pH clay or clay minerals, or by otherwise treating or altering the chemistry of a natural clay or clay mineral, to yield an antibacterial effective amount of a reducing agent within the clay mineral's crystal structure. In the latter preparation, for example, ferrous iron is incorporated into the octahedral sheet of a synthetic clay mineral, or it is introduced by the reduction of ferric iron already present in the octahedral sheet. Ferrous iron also could be introduced into the exchange positions of clay minerals (for example, into the interlayer position of smectitic clay minerals) by cation exchange.

The reducing agents suitable to render the compositions herein bactericidal are polymorphs of FeS2, which include pyrite and marcasite, but other similar agents may work as well, agents, such as manganese oxides, pyrrhotite, FeS, FeS04, and other minerals (natural or synthetic) or compounds that contain reducing agents that are present within the structure of the clay mineral itself. Reducing agents may be employed and be present in the compositions in the form of fine (submicron) particles. Their fine particulate sizes, as well as the amounts of these reducing agents present in the composition of the invention, do not materially affect the clay-like properties of the composition.

Natural and/or synthetic clays or clay minerals are employed in this composition, and serve as a carrier for the reducing agents. They bind/sorb the reducing agent and may play a role in buffering the chemical reaction(s) produced by the reducing agent(s). They also serve as an absorbent and low permeability barrier when the composition is in use. As well, the exchange properties of the clay mineral may enhance the solubility of sparingly soluble reducing agents, as has been found for the enhanced dissolution of other sparingly soluble compounds in the presence of montmorillonites (Eberl and Landa, 1985). The reducing agent imparts antibacterial properties to the clay containing compositions herein.

Smectite-clays, illite-clays, rectorite-clays and clays having like properties, or a combination of these, may be suitably employed as the carrier for the reducing agent in the present invention. These clays may be natural or synthetic. In addition to the suitability of natural and/or synthetic clays for use as the carrier herein polymers, for example, and other materials having clay-like properties (for example, kaolinites, chlorites) may be employed in this role as well. One having ordinary skill in the art with knowledge of the teachings herein would be readily able to identify the types of carriers that may be suitably employed herein. Moreover, one having ordinary skill in the art would recognize that natural clays may require processing so as to render them suitable for use.

Also, it may be necessary to remove accessory minerals, such as quartz, by particle size techniques (e.g. separation of quartz and feldspar from the clay minerals by settling in water or by centrifugation according to Stokes Law), and cation exchange to change the chemical, sorptive and swelling properties of the clay.

Applicants anticipate that one skilled in the art will also recognize that an antibacterial effective amount of a reducing agent may be added to any natural clay or clay mineral, regardless as to whether or not that natural clay or clay mineral has natural bactericidal properties, so as to be certain that a composition containing that natural clay is suitable for the purpose intended herein. Prior to the present invention, applicants contend that there was no motivation to add a reducing agent to a clay or clay mineral (natural or synthetic) for the use described herein.

Compositions within the scope of the present invention may be used to topically treat bacterial skin infections and diseases including those caused by antibiotic-resistant bacteria, such as methicillin resistant Staph. Aureus (MRSA). Water, or other suitable pharmaceutically acceptable aqueous liquids (e.g., inert solution), are added to a composition in sufficient amounts to create a paste. It is the composition in this paste form that is topically applied to an infected area, for example, of the skin. The mechanism in which these bactericidal compositions operate is different from the mechanism of commonly used or commercially available antibiotics.

Data from Williams et al. 2008 included results for two types of French clays; they indicated that the Argiletz French green clay was not bactericidal against E. coli. By contrast, Argicur supplied green clay displayed antimicrobial properties and was found to be bactericidal against several species of bacteria, including E. coli and S. aureus. One question was whether or not changing chemical conditions mediated by clays might be important in clay bacterial activity.

Experimentation with these clay types upon several bacterial types such as Salmonella, E. coli, Pseudomonas, Staphylococcus and Streptococcus, suggested that clay type and degree of bactericidal activity depended upon reducing conditions, possibly mediated by FeS2 (pyrite) or by other minerals with available reduced metals. Pyrite has been implicated in spontaneous production of chemical radicals; chemical radicals such as OH and 02-, would be highly damaging to biomolecules such as sugars, fatty acids or proteins located on bacterial cell surfaces and within cells. Additionally, the Fe2+ from pyrite might produce intracellular Fenton-type reactions (described later herein). The reaction products could damage nucleic acids such as DNA or RNA or hamper cellular metabolic functions. Blue clay (a clay from Oregon that naturally contains about 10% pyrite) was determined to be bactericidal, but neither weathered Blue clay (Blue clay that had been oxidized naturally by weathering) nor Ormalite (a commercial name for a clay from a nearby deposit) were particularly bactericidal. Of note is that neither weathered Blue clay nor Ormalite were found to contain pyrite or other reducing agents.

The reducing agent may be added to the natural clay or clay mineral, regardless as to whether or not that natural clay or clay mineral has natural bactericidal properties, so as to be certain that a composition containing that natural clay is suitable for the purpose intended herein. Prior to the present invention, there was no motivation to add a reducing agent to a high (>10) or low (<5) pH clay or clay mineral (natural or synthetic) for the use described herein.

### DETAILED DESCRIPTION

Various terms are used throughout the specification in describing the present invention. In order to provide a clearer and consistent understanding of the specification and claims, including the scope to be given such terms, the following definitions are provided.

Clay—In general, the term clay defines a natural, earthy, fine-grained material which develops plasticity when mixed with a limited amount of water. By plasticity is meant the property of the moistened material to be deformed under the application of pressure, with the deformed shape being retained when the deforming pressure is removed. Chemical analyses of clays show them to be composed essentially of silica, alumina, and water, frequently with appreciable quantities of iron, alkalis, and alkaline earths. (Grim, Clay Mineralogy, Second Edition, pp. 1-2 (1968)). The plasticity of clays generally results from a majority of their constituents having a very fine particle size (e.g., <2 micrometer equivalent spherical diameter). Some natural clays are composed of a single clay mineral, whereas others may contain a mixture of clay minerals. In addition, clays may contain non-clay minerals, such as quartz, feldspar, calcite, pyrite, organic material and salts. Natural and synthetic clays may be employed within the scope of the Invention. Reference herein to clays, unless specified differently, includes both natural and synthetic clays.

Clay minerals—Clay minerals are very fine-grained minerals (generally <2 micrometer equivalent spherical diameter) having the phyllosilicate structure. Because they are minerals, each type of clay mineral is defined by a certain crystal structure, which is, in the general case, the phyllosilicate-type structure, and each type of clay mineral has a limited range of chemical composition. A definition of the phyllosilicate structure (Bailey, 1980) states, Clay minerals belong to the family of phyllosilicates and contain continuous two-dimensional tetrahedral sheets of composition T205 (T-SI, Al, Be) with tetrahedra linked by sharing three corners of each, and with the fourth corner pointing in any direction. The tetrahedral sheets are linked in the unit structure to octahedral sheets, or to groups of coordinated cations, or individual cations. In other words, “clay minerals” are a class of fine grained chemical compounds having the phyllosilicate structure, whereas “clays,” by contrast, are defined by their physical properties (e.g., plasticity), although they most often contain a substantial amount of clay minerals. Natural and synthetic clay minerals may be employed within the scope of the Invention. Reference herein to clay minerals, unless specified differently, includes both natural and synthetic clay minerals.

Clay-like properties—Reference is made herein to materials having clay-like properties or the property of clays. For example, this phrase is used to describe the properties of the compositions within the scope of the Invention. For the purposes of the present invention, a composition or material having clay-like properties is one that is fine-grained (having a maximum particle size of approximately 2 microns), and that develops plasticity when mixed with a limited amount of water or other suitable aqueous solution.

Processed—This term is used with reference to natural clays and compositions containing natural clay in particular, and refers to the processing of these to render them suitable for new and novel use. For example, since natural clay samples are not pure clay minerals, processing may be necessary i.e., clay-sized particles of <2.0 μm in diameter might be collected, organic material removed, the samples washed and sterilized.

Paste—The term paste is used with reference to the form in which the composition within the scope of the present invention is used. The dry synthetic antibacterial clay composition within the scope of the present invention is hydrated to form a composition having the consistency of a paste. One skilled in the art would select a suitable amount of a pharmaceutically acceptable aqueous solution (i.e., water or saline) to add to the dry composition to provide a composition having the desired paste-like consistency. The specific consistency of the composition in the form of a paste is not critical so long as the hydrated composition, when in use, provides the desired properties described herein i.e., acts as a barrier to atmospheric oxygen preventing it from entering the treated area; acts to effectively disperse the reducing agent therein; etc.

Processed—This term is used with reference to natural clays and compositions containing natural clay in particular, and refers to the processing of these to render them suitable for new and novel topical pharmaceutical use. For example, since natural clay samples are not pure clay minerals, processing may be necessary i.e., clay-sized particles of <2.0 μm in diameter might be collected, organic material removed, the samples washed and sterilized using UV or heat to render any pathogenic virus, bacteria or protest non-Infectious.

Suitable aqueous liquid—This phrase is used with reference to a liquid that may be used for hydrating the compositions within the scope of the present invention to create the new and novel pastes thereof. It is intended that these liquids be pharmaceutically acceptable/suitable for the use intended herein. Use of water is preferred.

Applicants' study as to the antibacterial properties of many different natural clays resulted in their identifying a property which renders certain natural clays to have natural bactericidal properties. In particular, applicants identified that it is the presence of both high (>10) or low (<5) pH clay and a bactericidal amount of particular reducing agents (i.e., pyrite, marcasite, Fe2+) in these natural clays that renders them bactericidal. The effectiveness of the reducing agent is related to its solubility and reactivity, which, in turn, is influenced by its particle size. They have found what they consider to be a mechanism behind its action. Applicants are now able to artificially produce a bactericidal composition having clay-like properties (referred to herein as a synthetic bactericidal (or antibacterial) composition), wherein said composition can be customized so as to illicit the properties desired. These compositions may have some advantages over simply using a natural antibacterial clay. For example, the artificially produced clay composition can be made purer (i.e., use of synthetic pyrite in place of natural pyrite, because naturally occurring pyrite may contain arsenic, selenium, cadmium or other toxic impurities, and use of a synthetic smectite as opposed to a natural smectite, as it could be rendered free of toxic metals) than naturally occurring clays, and its properties (i.e., reducing agent dissolution rate) can be optimized for the type of Infection to be treated (I.e., the type of bacteria to be killed). Applicants' findings provide guidance useful in making synthetic bactericidal compositions having clay-like properties.

The present invention discloses synthetic bactericidal compositions having the properties of clay (clay-like properties), wherein these compositions comprise an bactericidal effective amount of a reducing agent and a high (>10) or low (<5) pH clay, clay mineral or material having clay-like properties, wherein both the reducing agent and the high (>10) or low (<5) pH clay present in the composition are responsible for rendering the composition bactericidal.

The Invention further discloses a method of topically treating bacterially-caused skin infections/diseases using these compositions. In use, water, or other suitable pharmaceutically acceptable aqueous liquids, such as sterile saline or phosphate buffered saline, is added to these compositions so as to create a paste (composition having a paste-like consistency). It is a composition in the form of a paste that is applied to the affected area for treatment.

Applicants have identified both high (>10) or low (<5) pH clay and reducing agents that are responsible for rendering compositions within the scope of the present invention bactericidal. Suitable reducing agents that may be employed herein are the polymorphs of FeS2, which include pyrite and marcasite, pyrrhotite, manganese oxides, FeS, FeSO4, and other minerals or compounds that contain soluble reducing transition metals with like properties. They are used to remove oxygen from the treated site and produce chemical radicals (i.e., hydroxyl, nitrogen, or oxygen). We speculate that peroxide may be produced at, for example, the pyrite surface, and that this peroxide participates in a Fenton reaction with the ferrous iron to produce hydroxyl radicals that then attack bacterial cell walls, thereby killing the bacteria. [References: Cohn et al., “Role of pyrite in formation of hydroxyl radicals in coal: possible implications for human health,” Particle and Fibre Toxicology, 3: 16 (2006); Cohn et al., “Pyrite-induced hydroxyl radical formation and its effect on nucleic acids,” Geochemical Transactions, 7:3 (2006); Cohn et al., “RNA decomposition by pyrite-induced radicals and possible role of lipids during the emergence of life,” Earth and Planetary Science Letters, 225, 271-278 (2004)].

These reducing agents are employed in the present invention in fine particulate form. Applicants have found that the finer the particle size, the more effective the reducing agents are, and the quicker the bactericidal activity occurs. Suitable particle sizes for the reducing agents herein may range from the nanometer to approximately 1 micron size range and preferably less than approximately 1 micron in size. In a preferred embodiment, it is best for the reducing agent to be well mixed/dispersed in the compositions herein.

The reducing agents are present in the compositions herein in a bactericidal effective amount. These amounts may range from approximately 0.5% wt. to 10% wt., and preferably about 3% wt. of said composition. Applicants have determined that the presence of trace amounts, even less than 0.1% wt., of pyrite, for example, may render the compositions herein bactericidal. Trace amounts of pyrite were detected in the original sample of Argicur clay used to successfully treat Buruli ulcer in the Ivory Coast, West Africa. Of note is that for a given particle size, the more pyrite, for example, present, the stronger the bactericidal effect; and for a given amount of pyrite generally the smaller the particle size, the stronger the bactericidal effect.

Because reactions involving, for example, pyrite oxidation may generate sulphuric acid, there is an optimal amount of pyrite, depending on its particle size, that may be present in a composition herein. With too much pyrite, the acid generated may mildly burn the skin tissue. For example, natural antibacterial clay from Oregon (Blue clay) was found to contain pyrite that is nano-size and contains approximately 10% by weight pyrite in bulk. This clay was found to be bactericidal and to produce fairly acidic leachates (H2SO4, pH 2-2.5). Application of this clay in an unbuffered solution might create a pH condition damaging to human or mammalian skin tissues. Therefore, although the addition of large quantities of a reducing agent such as pyrite, for example, into a composition such as that described herein would still render the composition effective as a bactericidal composition, the presence of larger quantities of pyrite would not be desirable. Addition of a pH buffer, such as calcite, to clay-pyrite compositions that will remove acidity but not significantly influence the invention's bactericidal activity, is within the scope of the present invention. In addition to the undesirable pH, the addition of large quantities of pyrite, for example, would also render the composition unpleasant to apply to the skin. The addition of pyrite could be avoided altogether if bactericidal effective amounts of reducing ions, such as Fe2+, for example, were incorporated directly into the clay mineral structure, or were present as exchange ions on the clay.

It is also within the scope of the present invention to incorporate one or more reducing agents in the compositions herein. Should more than one reducing agent be employed, one skilled in the art will recognize that the combined amount of these would be such as to have a combined bactericidal effect and, hence, the combined amount would translate as being a bactericidal effective amount present in the composition.

In a preferred embodiment, the fine particulate size, as well as the amounts of the reducing agent present in the composition of the invention, is such as to not materially affect the clay-like properties of the composition.

The preferred reducing agent is pyrite. Course grained, naturally occurring, pyrite may be ground and employed herein. As an alternative, pyrite can also be synthesized to be very fine. See Ohfuji et al., “Experimental syntheses of framboids—a review,” Earth Science Reviews, 71, pp. 147-170 (2005) and Shi et al., “Synthesis, characterization, and manipulation of dendrimer-stabilized iron sulfide nanoparticles,” Nanotechnology, 17, pp. 4554-4560 (2006). The teachings of these references are incorporated herein by reference.

High (>10) or low (<5) pH clays serve in the compositions herein as pharmaceutically acceptable carriers for the reducing agent. In addition to serving as a suitable carrier for the reducing agent, wherein the reducing agent may be thoroughly dispersed therein, the clays herein also (a) serve as an a moderate bactericidal agent due to the either high (>10) or low (<5) pH, (b) serve as an effective low permeability barrier to keep atmospheric oxygen from the skin or other surface to be treated, (c) serve to render compositions within the scope of the invention to have clay-like properties, (d) serve to keep the site/system to be treated moist, while absorbing excess liquid, and (e) aid in the dissolution of the reducing agent through the process of ion exchange, an effect that is described for the dissolution of sparingly soluble compounds by montmorillonite in Eberl and Landa, “Dissolution of alkaline earth sulfates in the presence of montmorillonite,” Water, Air, and Soil Pollution, 25: 207 (1985). The teachings of this reference are incorporated herein by reference.

It is also within the scope of the present invention that the final synthetic bactericidal composition have the properties of a clay. Suitable clays that may be employed herein include smectite-clays, illite-clays, rectorite-clays, other clays having like properties, or mixtures thereof. These clays may be natural or synthetic. In addition to clays, applicants contend that polymers or other materials having clay-like properties (fine particle size, and plasticity) may be suitably employed for the clays herein. One having ordinary skill in the art will recognize the purpose for which the clays are employed; and, accordingly, will recognize other suitable materials that may be used in its place. Applicants contend that these materials are included within the compositions claimed herein.

Should a composition within the scope of the present invention comprise a natural clay, one having ordinary skill in the art will recognize that there may be a need to process the natural clay so as to render it suitable for the purpose described herein, topical pharmaceutical use. How to process natural clays so as to render them suitable for pharmaceutical use is well within the skill of the art.

We speculate that certain clay minerals which contain ferrous iron (Fe+2) in their structure may not need to have a reducing agent added to demonstrate bactericidal properties (e.g., some smectite clay minerals have elevated Fe+2 content, but no pyrite). The existence of such iron (or other reduced transition metal) in the clay itself may fulfill the role, for example, of the pyrite. Ferrous iron, for example, could be present in the octahedral sheet of the clay mineral, or as an exchange ion in its interlayer, and act as an effective reducing agent. Octahedral ferrous iron present in smectite would be close to the solution, because the smectite particles are so thin (approximately 1 nm), and therefore could participate in oxidation-reduction reactions.

Smectite is a general name used for swelling clay that has approximately 1-nm thick 2:1 layers (c-direction of unit cell) separated by hydrated interlayer cations which give rise to the clay's swelling. The “a” and “b” dimensions of the mineral are on the order of several microns. The layers themselves are composed of two opposing silicate sheets, which contain Si and Al in tetrahedral coordination with oxygen, separated by an octahedral sheet that contains Al, Fe and Mg in octahedral coordination with hydroxyls. These 2:1 layers (two tetrahedral sheets with an octahedral sheet in between) carry a net negative charge that is balanced by interlayer cations. Because the surfaces of the 2:1 layers are charged, they attract cations and water, which leads to swelling. The operational definition is that smectite swells to give a 17 Angstrom-unit x-ray diffraction peak when treated with ethylene glycol. Because the 2:1 layers are less than 1 nm thick, and because the interlayer is open to solution, smectite has very special properties. For example, it has an enormous surface area of approximately 750 m2 per gram, can absorb polar organic and other molecules in the Interlayers, and can exchange interlayer cations with the solution. It can further protect interlayer organics from bacteria and oxygen, and can aid in the dissolution of insoluble substances through its exchange properties. Due to its very fine particle size, it can be impermeable to gases. In addition, it has catalytic properties important for many organic reactions as well.

There are a number of different types of smectite, which are classified with respect to the location of the negative charge on the 2:1 layers, and based on the composition of the octahedral sheet (either dioctahedral or trioctahedral). Dioctahedral smectites include beidellite (majority of charge located in tetrahedral sheet) and montmorillonite (majority of charge in octahedral sheet). Similar trioctahedral smectites are saponite and hectorite. Swelling and other properties of smectite can be altered by exchanging the dominant interlayer cation. For example, swelling can be limited to 2 water layers by exchanging Na for Ca.

Smectites are well known in the art and are available commercially from a variety of sources for example, from WyoBen Co. (Greybull, Wyo.) and American Colloid (now called AmCol, Arlington Heights, Ill.). In addition, they can be synthesized, and can be found in large deposits, particularly in the American western deserts i.e., the Cheto clay in Arizona. Smectites are frequently found in nature mixed with impurities (i.e., calcite and quartz), but, due to the very fine particle size, can be purified by size fractionation in water. During this process, the coarser grained minerals (i.e., quartz and calcite) settle out, and the smectite is poured off with the aqueous suspension and dried.

Numerous methods exist for synthesizing smectite. F or example, U.S. Pat. No. 4,861,584 (Powell, et al.) sets forth a description of smectite-type days (see col. 4, lines 37 through col. 5, line 55) and identifies that smectite clays can be prepared synthetically by either a pneumatolytic or a hydrothermal synthesis process. Powell et al. provides a list of USPs as describing representative hydrothermal processes for preparing synthetic smectites. USP '584 further describes at col. 5, lines 40+ a hydrothermal process for synthesizing smectite clays. The teachings set forth in Powell, et al., as well as the teachings set forth in the list of USPs identified in Powell et al., are incorporated by reference herein in their entirety.

It is within the scope of the present invention to utilize the various types of smectite in the compositions of the present invention.

It is also within the scope of the present invention to utilize illite-clay in the compositions of the present invention. Illite, though similar to smectite, is a non-swelling clay. It has its 2:1 layers bound together by K ions so that it does not swell.

Oregon Blue clay, for example, is composed of an ordered mixed-layer illite/smectite (referred to as K-rectorite). It has regular alternation of illite and smectite layers parallel to the “c” axis. It combines the swelling properties of smectite with non-swelling illite.

Suitable clays, for example, such as smectite, illite, mixed layer clays such as rectorite, and other suitable clays may be synthesized using conventional methods well known in the art. In general, one skilled in the art would simply start with a gel or glass having the chemical composition of the clay to be synthesized, and heat it in water using a pressure vessel. (For example, experimental variables, such as temperature, time and pressure required to synthesize smectite and various forms of rectorite are described in Eberl, “Reaction series for dioctahedral smectites,” Clays and Clay Minerals, Vol. 26, 327-340 (1978)). Suitable clays should be very fine grained, hold water well, and be absorptive.

Examples of synthetic clays and clay minerals that may be suitable for use herein include, but are not limited to, synthetic hectorite, which is a layered hydrous magnesium silicate known as Laponite® (Southern Clay Products, Gonzales, Tex.), a synthetic mica-montmorillonite, such as Bara-sym® (Baroid Division, NL Industries, Houston, Tex.) and mixtures thereof. Useful natural types of clays include swelling clays such as aliettite, beidellite, nontronite, saponite, sauconite, stevensite, swinefordite, volkonskoite, yakhontovite, hectorite, montmorillonite, bentonite and mixtures thereof. [Note U.S. Pat. No. 6,015,816 (i.e., col. 4, lines 10+) the teachings of USP '816 are incorporated by reference. This U.S. Pat. No. 6,015,816 also references examples of specific types of clays from the smectite mineral group as including: hectorite (“SHCa-1” Source Clay Minerals Repository, University of Missouri, Columbia, Mo.), Cheto montmorillonite (“SAz-1”), etc.]. In addition, 1:1 clay minerals such as kaolinite and serpentine, as well as 2:1:1 clay minerals such as chlorite may also prove to be useful, as may chain-type clays such as palygorskite and seplolite.

Selection of clays and/or materials having clay-like properties that would be suitable for use in the present invention is well within the skill of the art in light of the description herein.

Compositions within the scope of the present invention may be made by mixing one or more selected reducing agents with a selected clay, clay mineral or material having clay-like properties so as to disperse the reducing agent(s) therein. Ideally, the reducing agent(s) is uniformly dispersed throughout the synthetic composition.

The compositions herein are believed useful to topically treat infections and skin diseases caused by various types of bacteria such as, for example, Staphylococcus aureus (MRSA and non-MRSA), Pseudomonas aeruginosa, Streptococcus sp., Mycobacterium ulcerans, E. coli, ESBL E-coli, Salmonella typhimurium, Staphylococcus epidermidis, M. smegmatis, and M. marinum. Treatment of these infections is accomplished by hydrating the composition herein so as to form a paste, and then topically applying the paste to the affected area as well as to portions of the surrounding area. More than one, and perhaps a series of applications may be needed to treat the affected area. A clay compress, poultice, or paste should be washed off and/or changed daily. In addition, applicants believe that the compositions herein may also possibly be useful to treat protest skin infections such as Leishmania, and possibly viral skin infections as well.

The presence of reducing agents, such as pyrite, in the synthetic bactericidal compositions of the Invention is essential for rendering said composition bactericidal along with a high (>10) or low (<5) pH clay. At this point, applicants can only speculate as to the role played by the clays described herein. Applicants postulate that these types of clays can (1) absorb toxins, (2) absorb Fe+2 dissolved from the reducing agent (i.e., pyrite), (3) perhaps absorb and preserve H202 released by the Fenton reaction, and (4) keep oxygen away from the reaction so that Fe+2, for example, from the reducing agent, is not immediately oxidized, and (5) provide moderate antimicrobial activity through pH level (either above 10 or below 5), as most bacteria prefer neutral pH (6.5-7.0), while most molds and yeasts prefer pH between 5 and 6. (See “Proposed Theory of Activity,” outlined below.) In addition, the clays or materials having clay-like properties of the type described herein are excellent carriers for the reducing agents, are normally very soothing to the skin, and able to keep the system moist because they absorb water.

The reducing agent and pH level of the clay are essential to the composition of the present invention. Without wishing to be bound by theory, it is applicants' belief that the compositions within the scope of the present invention are antibacterial due to perhaps one, or more of the following activities:

(a) the removal of oxygen, depriving the bacteria of oxygen;

(b) the production of peroxide at the surface of the reducing agent, i.e., pyrite; and/or

(c) the production of hydroxyl radicals (.OH) by the Fenton reaction;

(d) clays or materials with having clay-like properties with pH level either above 10 or below 5.

Applicants believe that some of these activities are related to the presence of Fe+2, for example, in the reducing agent (i.e., pyrite) and in some cases other reduced forms of transition metals (e.g., Al, Mn, Cr, Zn, Cu) may substitute and produce a similar antibacterial reaction. It is believed that the invention deprives the bacteria of oxygen and may produce hydroxyl radicals, thereby causing disruption of bacterial cell membrane proteins and lipopolysaccharides. Applicants have learned that the finer the particle size of pyrite, for example, the faster the reaction-oxygen is removed faster and more completely. As well, the applicants believe that a high (>10) or low (<5) pH clay can inhibit certain microbial growth as most bacteria prefer neutral pH (6.5 to 7), while molds and yeasts prefer pH between 5 and 6.

A possible mechanism of how it is believed this theory might operate can be described as follows in relation to the use of pyrite as the reducing agent:

Some of the pyrite present in the composition oxidizes so as to remove oxygen from the system. Dissolution of the pyrite buffers the oxygen at a low level. It is believed that the clays or materials having clay-like properties described herein (i.e., smectite clay), and in which the pyrite is dispersed, prevent additional oxygen (atmospheric oxygen) from entering the system, and hence not allowing replacement of the oxygen that has been removed by the above reaction. By varying the clay composition, for example, the entry of oxygen into the system can be modified or controlled and the proposed reactions (see below) maintained. In addition, the clay itself may further sorb toxins released by the bacteria. With essentially no oxygen in the system, water present in the clay reacts at the pyrite surface to form peroxide (H202). It is believed that the reaction of water at the pyrite surface keeps H2O2 at a steady concentration. Fe+2 from the pyrite then reacts with the peroxide to form hydroxyl radicals (.OH) by the Fenton reaction (see Eq. 1 and Eq. 2):

Fe+2+H2O2→Fe+3+.OH+OH— (Eq. 1)

Fe+3+H2O2→Fe+2+OOH.+H+ (Eq. 2)

In the net reaction, the presence of iron is truly catalytic and two molecules of hydrogen peroxide are converted into two hydroxyl radicals and water-description obtained from http://en.wikipedia.org/wikl/Fenton's reagent.

Applicants believe that Fe+2 keeps up the steady production of hydroxyl radicals. It is further believed that these hydroxyl radicals then react with the bacterial cell wall, hence killing the bacteria. Within this theory, it is believed important that the peroxide (H2O2) be released slowly so as to not decompose into oxygen. The low oxygen level is buffered by the reducing agent; therefore, if additional oxygen enters the system, the reducing agent serves to remove it. Likewise, the peroxide may be buffered by reaction at the pyrite surface. It is believed that the pH of the composition should optimally range from above 10 to below 5. However, if the pH of the composition were to be within the range of 5 to 10, it would likely be difficult to maintain iron in the ferrous (Fe+2) state, and peroxide may decompose to oxygen.

In addition to clays such as smectite serving to prevent atmospheric oxygen from entering the system, the clay serves to keep the system moist. This is believed to be important because water is needed to produce the peroxide as set forth above. Applicants further believe that the clays or materials having clay-like properties may, perhaps, also absorb toxins. Clay minerals also may attract Fe2+ from the pyrite and hold it as an exchange ion, thereby rendering it more reactive than when it is held in the pyrite structure.

Described more generally, it is believed that the mechanism(s) of action appear to be bacterial death or viability loss caused by a lowered oxidation-reduction potential (reducing conditions), a several to many fold reduction in dissolved oxygen level, and/or a concomitant lowered pH. Applicants have found that bacteria viability was affected by (1) variation in pyrite content, (2) pyrite grain/particle size, and (3) degree of redox potential.

The reaction takes place in a matter of hours and reaches steady state within 24 hours. Perhaps it is the gradient, meaning the rapid oxidation-reduction potential (ORP) change over time that the bacteria cannot accommodate.

A method of producing the various compositions involves quarrying the clay, air drying the day until it has a moisture content of 20% to 25% by weight. The day is subsequently crushed and Introduced to a rotary dryer and blended with the remaining ingredients of the composition. The composition remains in the rotary dryer until it has a moisture content between 10% and 15% by weight. Water, or other suitable pharmaceutically acceptable aqueous liquids (e.g., inert solution), are added to a composition in sufficient amounts to create a paste. The hydrated composition is then applied topically to the site of the bacterial skin infection.

### Specific Formulations

Typical specific formulations are set forth below. It will be recognized and understood also that these embodiments are provided herein merely as illustrations of the many different formulations and compositions which may be usefully employed with high (>10) or low (<5) pH clays and reducing agents to provide antibacterial topical ointment preparations. Those skilled in the art will recognize that the specific ingredients recited and their relative amounts can be varied widely while still making available the benefits provided by the present invention.

The composition of the present invention is manufactured principally with two ingredients: clay with a high (>10) or low (<5) pH and a reducing agent, such as pyrite, FeS2.

The high (>10) or low (<5) pH of the clay imparts antibacterial properties which create an environment which is unsuitable for bacterial growth. Clays employed in this composition serve as a carrier for the reducing agents and may play a role in buffering the chemical reaction(s) produced by the reducing agent(s). As well, the exchange properties of the clay mineral may enhance the solubility of sparingly soluble reducing agents, as has been found for the enhanced dissolution of other sparingly soluble compounds in the presence of montmorillonites.

Clays may require processing so as to render them suitable for use and it may be necessary to remove accessory minerals, such as quartz, by particle size techniques (e.g. separation of quartz and feldspar from the clay minerals by settling in water or by centrifugation according to Stokes Law), and cation exchange to change the chemical, sorptive and swelling properties of the clay.

The percentage of bentonite in the composition can be reduced to a specified amount and replaced by filer materials, such as sand, shale or limestone. The higher percentage clay mixtures are more absorbent but are also more expensive than the lower percentage clay mixtures. Increasing the amount of filler material can serve to lower the overall cost of the composition. The increase of filler materials may reduce the effectiveness of the composition, therefore, the amount of fillers should be kept low.

Reducing agents suitable to render the compositions bactericidal are polymorphs of FeS2, which include pyrite and marcasite, but other similar agents may work as well, agents, such as manganese oxides, pyrrhotite, FeS, FeS04, and other minerals (natural or synthetic) or compounds that contain reducing agents that are present within the structure of the clay mineral itself.

The preferred reducing agent for the composition is pyrite, FeS2, as it is a common mineral and readily available as it is found in a wide variety of geological formations. Pyrite is a more economical choice for a reducing agent in the compositions herein. The reducing agents are present in the compositions herein in a bactericidal effective amount. These amounts may range from approximately 0.5% wt. to 10% wt., and preferably about 3% wt. of said composition. Applicants have determined that the presence of trace amounts, even less than 0.1% wt., of pyrite, for example, may render the compositions herein bactericidal. Of note is that for a given particle size, the more pyrite, for example, present, the stronger the bactericidal effect; and for a given amount of pyrite generally the smaller the particle size, the stronger the bactericidal effect.

Reducing agents may be employed and be present in the compositions in the form of fine (submicron) particles. Their fine particulate sizes, as well as the amounts of these reducing agents present in the composition of the invention, do not materially affect the clay-like properties of the composition.

The preparation which is the present invention permits the user to optionally include a variety of other ingredients as additives which are not essential—but may in various instances be desirable for that embodiment and Intended use circumstance. All of these additives are entirely optional; all of these additives are conventionally known and frequently employed in both a personal and commercial manufacture; and all of these provide properties which, under specific use circumstances, offer added benefits and advantages for that particular situation.

These additives, if employed, are generally present in small percentage quantities, typically 0.01-10.0 percent; and do not meaningfully influence or affect the admixture of essential component ingredients. Accordingly, the presence or absence of these optional additives is clearly a matter of choice; and any and all additives employed for this purpose within the preparations described herein are deemed to be within the scope of the present invention.

The present invention can also comprise additional ingredients such as perfumes, deodorants, odor absorbents and colorants.

### Example 1

An antimicrobial composition is prepared from the following ingredients:

### Ingredients Volume %

Calcium Bentonite 50 to 80% (high (>10) or low (<5) pH)

### Water 15% to 25%

### Shale 4%

### Pyrite 1%

### TOTAL 100%

Samples of each mixture were prepared by blending the materials. Any optional. Ingredients and additional absorbents are blended into the above ingredients of the present invention.

The resulting composition can be used as a topical ointment.

### Example 2

An antimicrobial composition is prepared from the following ingredients:

### Ingredients Volume %

Sodium Bentonite 15 to 25% (high (>10) or low (<5) pH)

### Water 65 to 80%

### Pyrite 5%

### TOTAL 100%

To use the present invention, user simply opens the product from its sealed packaging and apply a suitable volume thereof to the site of said bacterial skin infection

The dry clay is mixed with water and applied as a paste, poultice, directly to the skin of Infected patients.

However, depending on the type of clay, poultice consistencies used for therapeutic purposes are generally ratios of 1:2 or 1:3 (clay:water).

The mode of use of the composition and the formulation, that is the effective amount of the product to be applied depends on several factors: the area to be treated, the general conditions of the subject, the type of pathology and/or etiology underlying the inflammation.

An “effective amount” means an amount necessary at least partly to attain the desired response, or to delay the onset or inhibit progression or halt altogether, the onset or progression of a particular condition being treated. The amount varies depending upon the health and physical condition of the Individual to be treated, the taxonomic group of individual to be treated, the hairiness of the individual to be treated, the degree of protection desired, the formulation of the composition, the assessment of the medical situation, and other relevant factors. It is expected that the amount will fall in a relatively broad range that can be determined through routine trials.

The pH of the aqueous poultice material is adjusted typically, to a range of 3.0 to 5.0, preferably to a range of 3.5 to 5.0, and more preferably to a range of 4.0 to 5.0. The poultice having a pH lower than 3.0 is excessively acidic and thus can cause a significant skin irritation.

The topical medical formulations are suitable for treating mammals, including humans. The term “mammal” as used herein includes humans, racing animals (eg. horses or dogs), primates, livestock animals (eg. sheep, pigs, cattle, horses, donkeys), laboratory test animals (eg. mice, rabbits, rats, guinea pigs), companion animals (eg. dogs, cats) and captive wild animals (eg. foxes, kangaroos, deer).

To maintain in situ the composition and/or the formulation and depending on the preferred embodiment, a support can be used, for example a gauze, a portion of non-woven fabric or absorbent paper, taking care that the support always maintains a sufficient quantity of the formulation according to the present invention on the skin.

The essential components for the antibacterial activity of the antibacterial clay are Fe2+ and Fe3+(from the reducing agent), which work synergistically to overcome the highly evolved metabolic functions of human pathogens. The hydrated antibacterial clays generate a low pH (<4.6) environment, either inherently or through mineral oxidation, dissolution and hydrolysis reactions, sustaining metal release and reactive oxygen species (“ROS”) production throughout the antibacterial mechanism. Expandable smectite interlayers provide a reservoir for Ca2+ and Fe2+ exchange. Pyrite oxidation by dissolved O2 generates H2O2 on the mineral surfaces, while oxidation by Fe3+ releases Fe2+ into solution. Hydroxyl radicals are generated as Fe2+ is oxidized to Fe3+ by H2O2, providing additional Fe3+ for pyrite oxidation. Hydrolysis of Fe3+ generate H+, maintaining low pH (<4.6), promoting mineral dissolution and metal solubility.

The reactions generate Fe2+ that synergistically attack pathogenic bacteria in an oxidizing, metal rich environment that damages multiple cellular components. Fe2+ accumulate on cell envelopes, impairing membranes through oxidation (Fe2+), activating the σE-stress response. Cell envelopes remain enriched in Fe2+, providing a constant source of .OH attack on the cell envelope. Fe2+ floods the cytoplasm generating intracellular .OH that damages DNA and proteins, marking oxidation sites with Fe3+-oxide precipitates. This constant intracellular oxidation causes single strand DNA breaks and overwhelms the cells defense mechanisms against ROS.

In order to confirm the efficacy of the composition set out herein, some preliminary tests have been carried out on three samples and detected no microbiological activity through standard paper disc testing using the disc diffusion method.

1. A synthetic bactericidal composition for use in promotion of healing of a bacterial infection on the skin of a patient, comprising a bactericidal effective amount of a particulate reducing agent selected from the group consisting of pyrite, marcasite, pyrrhotite, FeS2, FeS, FeSO4, and a combination thereof, and a high (>10) or low (<5) pH clay or clay mineral comprising a smectite clay, an illite clay, a rectorite clay, or a combination thereof.

2. The composition of claim 1, wherein said fine particulate reducing agent is present in said composition in an amount ranging from approximately 0.5% wt. to 10% wt. of said composition

3. The composition of claim 1, wherein the particle size of said reducing agent is less than 1 micron.

4. The composition of claim 1, wherein said reducing agent is pyrite, and wherein said clay is a smectite clay.

5. The composition of claim 4, wherein said composition comprises 0.5% wt. to 10% wt. fine particulate pyrite; and wherein the particle size of said pyrite is less than one micron.

5. The composition of claim 4, containing a suitable aqueous liquid in an amount so as to create a paste.

6. The composition of claim 5, wherein said paste is arranged to be topically applied to the site of said bacterial skin infection.

7. A method of promoting healing of a bacterial skin infection on the skin of a patient, comprising topically applying to said bacterial skin infection a composition comprising a bactericidal effective amount of a particulate reducing agent selected from the group consisting of pyrite, marcasite, pyrrhotite, FeS2, FeS, FeSO4, and a combination thereof, and a high (>10) or low (<5) pH clay or clay mineral comprising a smectite clay, an illite clay, a rectorite clay, or a combination thereof.

8. The method of claim 7 comprising adding to the composition a suitable aqueous liquid in an amount so as to create a paste.

9. The method of claim 8 comprising topically applying said hydrated composition to the site of said bacterial skin infection.

10. The method of claim 8 wherein said suitable aqueous liquid is water.

11. The method of claim 7, wherein said bacterial skin infection is caused by one or more bacteria selected from the group consisting of Mycobacterium ulcerans, E. coli, ESBL E. coli, Pseudomonas aeruginosa, Salmonella typhimurium, Staphylococcus epidermidis, S. aureus, MRSA, M. smegmatis, Streptococcus sp. and M. marinum.

12. The method of claim 7, wherein said fine particulate reducing agent is present in said composition in an amount ranging from approximately 0.5% wt. to 10% wt. of said composition

13. The method of claim 7, wherein the particle size of said reducing agent is less than 1 micron.

14. The method of claim 7, wherein said reducing agent is pyrite, and wherein said clay is a smectite clay.

15. The method of claim 14, wherein said composition comprises 0.5% wt. to 10% wt. fine particulate pyrite; and wherein the particle size of said pyrite is less than one micron.

16. The method of claim 7, wherein the synthetic antibacterial clay or clay mineral is produced by: synthesizing a clay or clay mineral or by treating or altering the chemistry of a natural clay or clay mineral to yield a synthetic antibacterial clay or clay mineral containing within its crystal structure or within its exchange positions an antibacterial effective amount of a reducing agent; wherein said reducing agent ferrous iron and renders said clay or clay mineral antibacterial.
